(19) United States
(12) Patent Application Publication
(10) Pub. No.: US 2022/0142483 A1
(43) Pub. Date: May 12, 2022

(54) MEDICAL FLUORESCENCE IMAGING DATA ACQUISITION SYSTEM AND ACQUISITION METHOD THEREOF

(71) Applicant: Nanjing Nuoyuan Medical Devices Co., Ltd., Nanjing (CN)

(72) Inventors: Huiming Cai, Nanjing (CN); Changliu Li, Nanjing (CN); Kena Ni, Nanjing (CN)

(21) Appl. No.: 17/394,178

(22) Filed: Aug. 4, 2021

(30) Foreign Application Priority Data
Nov. 6, 2020 (CN) 202011230716.2

(51) Int. Cl. A61B 5/00

(52) U.S. Cl. CPC A61B 5/0071; A61B 5/413

(57) ABSTRACT

The embodiment of the invention discloses a medical fluorescence imaging data acquisition system and an acquisition method thereof and belongs to the technical field of fluorescence imaging. The medical fluorescence imaging data acquisition system according to the invention comprises a shooting management module, a fluorescence characteristic storage module, a fluorescence region query module, a fluorescence data acquisition module and an image detection module, wherein the shooting management module is used for controlling and acquiring image information of fluorescence regions by adjusting a focal length, and the fluorescence characteristic storage module is used for storing comparative fluorescence characteristic information in the image information. When the present invention is used for acquiring fluorescence region images at fluorescence positions, by determining and comprising the fluorescence quantity and area information of each fluorescence region with standard data, the fluorescence image is shot again after a shooting focal length is adjusted, to acquire the clearer fluorescence image and reduce later image screening work.

### CROSS-REFERENCE TO RELATED APPLICATIONS

This application claims priority to Chinese Patent Application No. 202011230716.2 with a filing date of Nov. 6, 2020. The content of the aforementioned applications, including any intervening amendments thereto, are incorporated herein by reference.

### TECHNICAL FIELD

The embodiment of the present invention relates to the technical field of fluorescence imaging, in particular to a medical fluorescence imaging data acquisition system and an acquisition method thereof.

### BACKGROUND

Fluorescence is a common luminescence phenomenon in nature. The fluorescence is generated by interaction of photons with molecules, and the interaction process can be described by a Jablonslc molecular energy diagram as follows: most molecules are in the lowest vibration energy level So of a ground state in a normal state, the electrons around atomic nuclei are transited from the ground state energy level So to an excited state (a first excited state or a second excited state) with relatively high energy after being excited by energy (light energy, electric energy, chemical energy and the like), and the electrons in the excited state are in a high-energy state and unstable and may return to the ground state after releasing the energy through two ways, wherein one way is a radiative transition (including fluorescence and phosphorescence processes) releasing the energy in a form of photons, and the other way is a non-radiative transition releasing the energy in a form of heat energy and the like. Generally, after being excited and transiting from the ground state So to the excited state Si, the electrons outsides the atomic nuclei may rapidly fall at the lowest vibration energy level in a mode of non-radiative transition and then return to the ground state from the lowest vibration energy level to release the energy in the form of photon radiation, and emergent light with the property is called fluorescence.

In the acquisition process of existing medical fluorescence images, fluorescence images are acquired in a mode of direct shooting generally, and the quantity of data acquired in such mode is large; and the clear images can be obtained only after the fluorescence region quantity and the fluorescence region areas are in a reasonable range in available images, so that screening is relatively toilsome and very inconvenient.

Based on this, the present invention designs the medical fluorescence imaging data acquisition system and the acquisition method thereof to solve the above problems.

### SUMMARY

The embodiment of the invention provides a medical fluorescence imaging data acquisition system and an acquisition method thereof to solve the technical problems mentioned in the background.

The embodiment of the invention provides the medical fluorescence imaging data acquisition system. In one feasible solution, the medical fluorescence imaging data acquisition system comprises a shooting management module, a fluorescence characteristic storage module, a fluorescence region query module, a fluorescence data acquisition module and an image detection module;

The shooting management module is used for controlling and acquiring the image information of fluorescence regions by adjusting a focal length;

The fluorescence characteristic storage module is used for storing comparative fluorescence characteristic information in the image information;

The fluorescence region query module is used for querying fluorescence regions which have specified similarity with the comparative fluorescence characteristics in the acquired images;

The fluorescence data acquisition module is used for acquiring quantity information of the fluorescence regions and area data information of the fluorescence regions in the fluorescence images;

The image detection module is used for comparing quantity and area information of the acquired fluorescence regions with the standard data, controlling the shooting management module to adjust and control the focal length and outputting a standard image result.

The embodiment of the invention provides a medical fluorescence imaging data acquisition system. In one feasible solution, the shooting management module comprises an image shooting module and a focal length adjusting module;

The image shooting module is used for shooting the images at the fluorescent positions;

and the focal length adjusting module is used for receiving a control signal of the image detection module and automatically adjusting the shooting focal length.

the embodiment of the invention provides a medical fluorescence imaging data acquisition system. In one feasible solution, the fluorescence data acquisition module comprises a fluorescence quantity extraction module and a fluorescence region area measurement module;

The fluorescence quantity extraction module is used for extracting the quantity information of the fluorescence regions in the queried images; and

The fluorescence region area measurement module is used for measuring and calculating area data information of all the fluorescence regions.

The embodiment of the invention provides a medical fluorescence imaging data acquisition system. In one feasible solution, the image detection module comprises a standard data storage module, a comparative control module and an image output module;

The standard data storage module is used for storing standard fluorescence data information which is compared with the acquired fluorescence data;

The comparative control module is used for comparing the fluorescence data acquired by the fluorescence data acquisition module with the standard data and automatically controlling the shooting management module to be adjusted and controlled; and

and the image output module is used for outputting the acquired image information when the acquired fluorescence data and the standard fluorescence data reach the specified similarity.

The embodiment of the invention provides a medical fluorescence imaging data acquisition system. In one feasible solution, the standard data storage module comprises a standard fluorescence quantity data storage sub-module and a standard fluorescence area storage sub-module;

The standard fluorescence quantity data storage sub-module is used for storing standard fluorescence quantity data information; and

The standard fluorescence area storage sub-module is used for storing standard fluorescence region area data information.

The embodiment of the invention also provides a medical fluorescence imaging data acquisition method. In one feasible solution, the method comprises the following steps of:

S1, shooting a fluorescence position and acquiring fluorescence images;

S2, querying information of fluorescence region areas and a fluorescence region quantity of the fluorescence images through the fluorescence characteristics; and

S3, judging the similarity between the information of the fluorescence region areas and the fluorescence region quantity and the standard data to control adjustment on a focal length for image acquisition and controlling and outputting the fluorescence images when the specified similarity is reached.

Based on the solution, when the invention acquires the fluorescence region images at the fluorescence position.by determining and comprising the fluorescence quantity and area information of each fluorescence region with standard data, the fluorescence image is shot again after a shooting focal length is adjusted, to acquire the clearer fluorescence image and reduce later image screening work.

### BRIEF DESCRIPTION OF DRAWINGS

In order to more clearly illustrate the embodiments of the invention or the technical solutions in the prior art, the drawings used in the embodiments or the description of the prior art will be briefly described below, and it is obvious that the drawings in the following description are some embodiments of the present invention, and those skilled in the art can obtain other drawings based on the drawings without inventive labor.

FIG. 1 is a system architecture diagram of the acquisition system according to the invention;

FIG. 2 is a system detail drawing of a standard data storage module according to the invention;

FIG. 3 is a flow chart diagram of an acquisition method according to the invention.

### DETAILED DESCRIPTION

In order to make objectives, technical schemes and advantages of the invention more clear, the technical solutions used in the embodiments according to the invention are clearly and completely described below by the drawings of the embodiments according to the invention. Obviously, the embodiments described are only a partial embodiments rather than all embodimentsBased on the embodiments of the present invention, all the other embodiments obtained by those of ordinary skill without any creative works are within the protection scope of the present invention.

In the description of the invention, it is to be understood that orientations and positional relationships indicated by the terms “central”, “longitudinal”, “lateral”, “upper”, “lower”, “front”, “rear”, “left”, “right”, “vertical”, “horizontal”, “top”, “bottom”, “inner”, “outer”, “axial”, “radial”, “circumferential” and the like are based on the orientation or positional relationship illustrated in the drawings for convenience in describing the present invention and to simplify the description, and are not intended to indicate or imply that the indicated device or element must have a particular orientation, be constructed and operated in a particular orientation, and are not to be construed to limit the invention.

In the present invention, unless otherwise specifically stated or limited, the terms “mounted”, “connected”, “fixed” and the like are to be construed broadly and may include, for example, fixed, detachable or integral connections, mechanical connection, electrical connection, communication connection, direct connection or indirect connection through an intervening medium, communication between two elements, or interaction between two elements, unless otherwise specifically stated or limited. The specific meanings of the above terms in the present invention can be understood according to specific situations by those of ordinary skill in the art.

The technical solution of the invention will be described in detail below through specific embodiments. These several specific embodiments may be combined with each other below, and details of the same or similar concepts or processes may not be repeated in some embodiments.

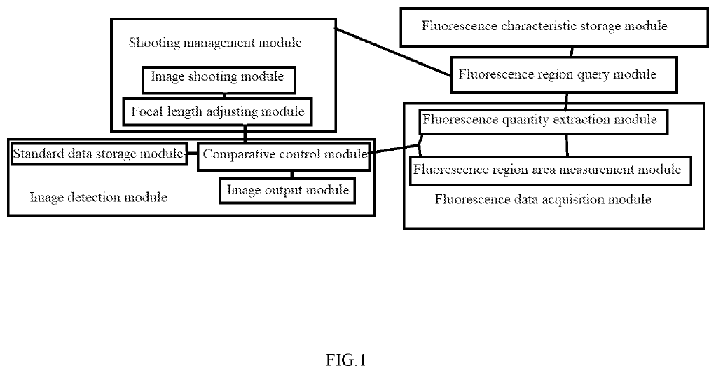

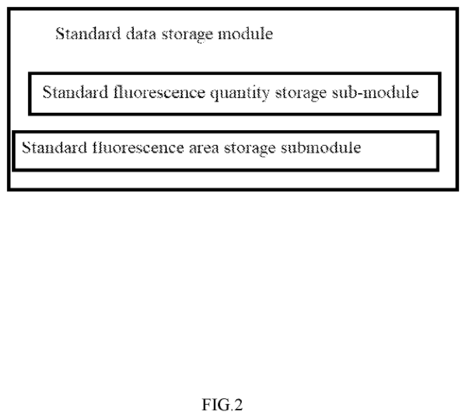

FIGS. 1-2 show a medical fluorescence imaging data acquisition system in Embodiment 1 of the present invention. The medical fluorescence imaging data acquisition system comprises a shooting management module, a fluorescence characteristic storage module, a fluorescence region query module, a fluorescence data acquisition module and an image detection module;

The shooting management module is used for controlling and acquiring image information of fluorescence regions by adjusting a focal length;

The fluorescence characteristic storage module is used for storing comparative fluorescence characteristic information in the image information;

The fluorescence area query module is used for querying the fluorescence regions which have specified similarity with the comparative fluorescence characteristics in the acquired images;

The fluorescence data acquisition module is used for acquiring the quantity information of the fluorescence regions and area data information of the fluorescence regions in the fluorescence images; and

The image detection module is used for comparing quantity and area information of the acquired fluorescence regions with standard data, controlling the shooting management module to adjust and control the shooting focal length and outputting a standard image result.

It is easy to find from the above description that when the medical fluorescence imaging data acquisition system according to the invention acquires fluorescence imaging data, zoom control may be achieved to shoot the fluorescence position by using the shooting management module, and shoot fluorescence images are queried about fluorescence regions with fluorescence characteristics in the fluorescence image storage module by the fluorescence region inquiring module, the fluorescence region area and the fluorescence region quantity in the fluorescence image are acquired by the fluorescence data acquisition module and then are introduced into the image detection module, and the fluorescence region area and fluorescence data are compared with standard data to adjust a shooting focal length and to obtain shoot images again, for example, when the fluorescence region areas are too large in the fluorescence images, the focal length can be adjusted to reduce the fluorescence region areas, so that relatively clear fluorescence images can be seen; and when the quantity of the fluorescence regions is relatively small, the areas of all the fluorescence regions are relatively dispersed, the areas of local fluorescence regions may be increased by adjusting the focal length, and the clearer fluorescence images can be achieved.

Alternatively, the shooting management module comprises an image shooting module and a focal length adjusting module;

The image shooting module is used for shooting images at the fluorescence position; and

The focal length adjusting module is used for receiving the control signal of the image detection module and automatically adjusting the shooting focal length. It should be noted that, in this embodiment, during the shooting management process, when the focal length is adjusted, the focal length is automatically adjusted by receiving a feedback signal from the image detection module, so that clearer image information may be obtained.

Further, the fluorescence data acquisition module comprises a fluorescence quantity extraction module and a fluorescence region area measurement module;

The fluorescence quantity extraction module is used for extracting the quantity information of the fluorescence regions in the queried images;

The fluorescence region area measurement module is used for measuring and calculating area data information of all the fluorescence regions; and when the fluorescence data are acquired, the fluorescence quantity and areas of the fluorescence regions are acquired, and thus the shooting focal length can be readjusted conveniently according to the information of the fluorescence quantity and the information of the fluorescence region areas.

More specifically, the image detection module comprises a standard data storage module, a comparative control module and an image output module;

The standard data storage module is used for storing standard fluorescence data information which is compared with the acquired fluorescence data;

The comparative control module is used for comparing the fluorescence data acquired by the fluorescence data acquisition module with standard data and automatically controlling the shooting management module to be adjusted and controlled;

The image output module is used for outputting acquired image information when the acquired fluorescence data reach the specified similarity with the standard fluorescence data; and during image detection, by comparing the acquired fluorescence quantity and the fluorescence region areas with the standard data, when a difference between the acquired data and the standard data is relatively large, the focal length can be readjusted for image shooting, so that the fluorescence region data in the images is in a reasonable range, and acquisition of the fluorescence images is achieved.

Further, the standard data storage module comprises a standard fluorescence quantity data storage sub-module and a standard fluorescence area storage sub-module;

The standard fluorescence quantity data storage sub-module is used for storing standard fluorescence quantity data information;

The standard fluorescence area storage sub-module is used for storing data information of a standard fluorescence region area; and in a standard data storage library, automatic adjustment and control work of the shot fluorescence image can be conveniently achieved by storing the standard fluorescence quantity and the standard fluorescence area data.

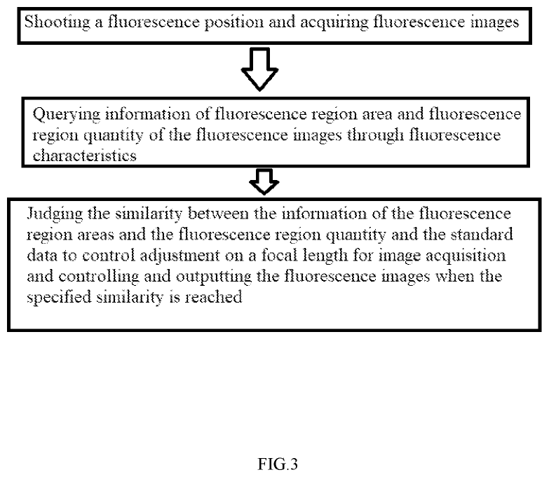

FIG. 3 shows a medical fluorescence imaging data acquisition method in Embodiment 2 of the present invention, and the Embodiment 2 is an improved solution based on Embodiment 1. The medical fluorescence imaging data acquisition method comprises the following steps:

S1, shooting a fluorescence position and acquiring fluorescence images;

S2, querying information of fluorescence region areas and a fluorescence region quantity of the fluorescence images through the fluorescence characteristics; and

S3, judging the similarity between the information of the fluorescence region areas and the fluorescence region quantity and the standard data to control adjustment on a focal length for image acquisition and controlling and outputting the fluorescence images when the specified similarity is reached.

Through the medical fluorescence imaging data acquisition method of the present invention, the automatic adjustment and control work of the acquired fluorescence images on the shooting focal length according to the fluorescence quantity and the fluorescence area information may be conveniently achieved, so that clear fluorescence image information meeting the requirements may be quickly and accurately acquired.

In the present invention, unless expressly stated or limited otherwise, that the first feature is “on” or “under” the second feature may be directly contacting the first feature with the second feature, or indirectly in contacting the first feature with the second feature through an intervening medium.

Moreover, that the first feature is “on”, “above” and “over” the second feature may represent that the first feature is right on or obliquely above the second feature, or merely means that the first feature is at a higher level than the second feature. That the first feature is “beneath”, “below” and “beneath” the second feature may represent that the first feature is right below or obliquely beneath the second feature, or merely means that the first feature is at a lower level than the second feature.

In the specification, with reference to the description of “one embodiment”, “some embodiments”, “an example”, “a specific example”, “some examples” or the like is intended to mean that a particular feature, structure, material, or characteristic described in combination with the embodiment or example is included in at least one embodiment or example of the present invention. In this specification, the schematic representations of the terms used above are not necessarily intended to refer to the same embodiment or example. Moreover, the particular feature, structure, material or characteristic described may be combined in any suitable manner in any one or more of the embodiments or examples. Further, various embodiments or examples and the features of various embodiments or examples described in this specification can be combined by one skilled in the art without contradicting each other.

Finally, it should be noted that: the above embodiments are only used to explain the technical solution of the present invention and shall not be construed as limitation. Although the present invention has been described in detail with reference to the foregoing embodiments, it should be understood by those skilled in the art that modifications may still be made to the technical solution described in the foregoing embodiments, or equivalent substitutions are made on a part of or the whole technical features; and these modifications or substitutions do not deviate the nature of the corresponding technical solution from the scope of the technical solution in respective embodiments of the present invention.

We claim:

1. A medical fluorescence imaging data acquisition system, characterized by comprising a shooting management module, a fluorescence characteristic storage module, a fluorescence region query module, a fluorescence data acquisition module and an image detection module;
the shooting management module is used for controlling and acquiring image information of fluorescence regions by adjusting a focal length;
the fluorescence characteristic storage module is used for storing comparative fluorescence characteristic information in the image information;
the fluorescence region query module is used for querying fluorescence regions which have specified similarity with the comparative fluorescence characteristics in the acquired images;
the fluorescence data acquisition module is used for acquiring quantity information of the fluorescence regions and the area data information of the fluorescence regions in the fluorescence image; and
the image detection module is used for comparing the quantity and area information of the acquired fluorescence regions with standard data, controlling the shooting management module to adjust and control the focal length for shooting and outputting a standard image result.

2. The medical fluorescence imaging data acquisition system according to claim 1, wherein the shooting management module comprises an image shooting module and a focal length adjusting module;
the image shooting module is used for shooting the images at the fluorescence position;
and the focal length adjusting module is used for receiving a control signal of the image detection module and automatically adjusting the focal length.

3. The medical fluorescence imaging data acquisition system according to claim 1, wherein the fluorescence data acquisition module comprises a fluorescence quantity extraction module and a fluorescence region measurement module;
the fluorescence quantity extraction module is used for extracting the quantity information of the fluorescence regions in the inquired image;
and the fluorescence region measurement module is used for measuring and calculating the area data information of all the fluorescence regions.

4. The medical fluorescence imaging data acquisition system according to claim 1, wherein the image detection module comprises a standard data storage module, a comparative control module and an image output module;
the standard data storage module is used for storing the standard fluorescence data information which is compared with the acquired fluorescence data;
the comparative control module is used for comparing the fluorescence data acquired by the fluorescence data acquisition module with the standard data and automatically controlling the adjustment and control of the shooting management module;
and the image output module is used for outputting the acquired image information when the acquired fluorescence data reach the specified similarity with the standard fluorescence data.

5. The medical fluorescence imaging data acquisition system according to claim 4, wherein the standard data storage module comprises a standard fluorescence quantity data storage sub-module and a standard fluorescence area storage sub-module;
the standard fluorescence quantity data storage sub-module is used for storing standard fluorescence quantity data information;
and the standard fluorescence area storage sub-module is used for storing the standard fluorescence data area data information.

6. An acquisition method for the medical fluorescence imaging data acquisition system according to claim 1, characterized by comprising the following steps:
S1, shooting a fluorescence position and acquiring fluorescence images;
S2, querying the information of fluorescence region areas and a fluorescence region quantity of the fluorescence images through the fluorescence characteristics;
and S3, judging the similarity between the information of the fluorescence region areas and the fluorescence region quantity and the standard data to control the adjustment of the focal length for image acquisition and controlling and outputting the fluorescence images when the specified similarity is reached
